(19) United States
(12) Patent Application Publication
(10) Pub. No.: US 2014/0023732 A1
(43) Pub. Date: Jan. 23, 2014

(54) USE OF A TRADITIONAL CHINESE MEDICINE COMBINATION IN PREPARATION OF ANTI-ENTEROVIRUS 71 DRUG

(71) Applicants: Hui Cao, Zhuhai (CN); Yi Guan, Zhuhai (CN); Yongqing Zeng, Zhuhai (CN); Desheng Tao, Zhuhai (CN)

(72) Inventors: Hui Cao, Zhuhai (CN); Yi Guan, Zhuhai (CN); Yongqing Zeng, Zhuhai (CN); Desheng Tao, Zhuhai (CN)

(73) Assignees: NATIONAL ENGINEERING RESEARCH CENTER FOR MODERNIZATION OF TRADITIONAL CHINESE, GUANGDONG PROVINCE (CN); LIVZON PHARMACEUTICAL GROUP, INC., GUANGDONG PROVINCE (CN)

(21) Appl. No.: 13/990,920

(22) PCT Filed: Dec. 28, 2011
(86) PCT No.: PCT/CN11/84847

§ 371 Date: Aug. 2, 2013

(30) Foreign Application Priority Data
Mar. 31, 2011 (CN) 201110079938.3

(51) Int. Cl. A61K 36/67; A61K 36/282; A61K 36/484; A61K 36/315

(52) U.S. Cl. CPC A61K 36/67; A61K 36/315; A61K 36/282; A61K 36/484
USPC 424/740; 424/725; 424/755; 424/757

(57) ABSTRACT

Disclosed is a use of a pharmaceutical composition in preparation of anti-enterovirus 71 agents.

This application claims the priority for the invention patent application submitted to State Intellectual Property Office of the People's Republic of China on Mar. 31, 2011 (its application number is 201110079938.3).

### FIELD OF THE INVENTION

This invention relates to the use of a traditional Chinese medicine combination in preparing of anti-enterovirus 71 drug, particularly the use of a traditional Chinese medicine combination containing Herba Houttyniae, Caulis Lonicerae, Radix Isatidis and Radix Sophorae Tonkinensis in preparation of anti-enterovirus 71 drug.

### BACKGROUND OF THE INVENTION

Enterovirus 71 virus (EV71) was isolated from the stool sample of infants with central nervous system disease in USA in 1969. Human enteroviruses are divided into five categories, namely A, B, C, D and new enterovirus (not classified) according to the latest classification by the International Committee on Taxonomy of Viruses (ICTV). Afterwards, the original serological types of enterovirus are re-classified, among which EV71 is classified as human enterovirus type A.

EV71 can be transmitted via digestive and respiratory tracts. Its infectivity and virulence are high, and its neurotoxicity is only secondary to that of poliovirus. Some patients eliminate viruses for several weeks, and the viruses can survive for a relatively long period in dirty water. People of different ages can be infected by this virus. Adults and elder children are mainly inapparent infection. Cases falling ill are mainly preschool children, and serious cases are mostly found in infants. It often leads to fulminant infection in local areas, and the reasons are still unknown. The clinical manifestations induced by EV71 are various: hand-foot and mouth disease and herpangina are the most common. Some of the cases may be characterized by aseptic meningitis and encephalitis, and the mortality of serious cases in infants is as high as 10%-25%.

Effective anti-virus drugs for EV71 infection are still absent, and the emphasis of prevention and treatments on the induced diseases is to research and develop effective preventive vaccines. Attempts on deactivated vaccines, attenuated vaccines, peptide or protein vaccines, DNA vaccines have been made, but no vaccine for EV71 has been put into market until now. Therefore, it is urgent to develop effective and safe anti-EV71 drugs.

### SUMMARY OF THE INVENTION

Therefore, the purpose of this invention is to provide a drug that can effectively inhibit EV71 and thereby successively prevent and/or treat the diseases induced by the virus.

The aim of this invention is achieved by the technical program as follows. Firstly, this invention provides a traditional Chinese medicine combination used for anti-EV71 or treatment and/or prevention of the diseases induced by EV71, and the said traditional Chinese medicine combination is prepared of the crude drugs as follows: Herba Houttyniae, Caulis Lonicerae, Radix Isatidis and Radix Sophorae Tonkinensis.

Preferably, the weight percentages of the four crude drugs in the said traditional Chinese medicine combination are: Herba Houttyniae 4.7%-70.0% in weight, Caulis Lonicerae 4.7%-46.7% in weight, Radix Isatidis 2.3%-42.0% in weight, Radix Sophorae Tonkinensis 1.2%-35.0% in weight.

Preferably, the said traditional Chinese medicine combination also contains other traditional Chinese medicines for eliminating heat evil and removing toxins; more preferably, the said traditional Chinese medicines for eliminating heat evil and removing toxins are selected from Cyrtomium fortunei, Radix Angelicae Dahuricae, Herba Andrographitis, Herba Taraxaci, Indigo Naturalis, Caulis Sargentodoxae, Rhizoma Paridis, Herba Artemisiae Annuae, Rhizoma Belamcandae, Brucea javanica, Herba Menthae Haplocalycis, Radix Platycodi and Radix Glycyrrhizae.

This invention also provides a traditional Chinese medicine combination used for anti-EV71 or treatment and/or prevention of the diseases induced by EV71, and the said traditional Chinese medicine combination is prepared of the crude drugs as follows: Herba Houttyniae, Caulis Lonicerae, Radix Isatidis, Radix Sophorae Tonkinensis, Cyrtomium fortunei and Radix Angelicae Dahuricae.

Preferably, the percentages by weight for the six crude drugs in the said traditional Chinese medicine combination are: Herba Houttyniae 4.7%-58.4% in weight, Caulis Lonicerae 4.7%-46.7% in weight, Radix Isatidis 2.3%-42.0% in weight, Radix Sophorae Tonkinensis 1.2%-35.0% in weight, Cyrtomium fortunei 1.9%-37.3% in weight, Radix Angelicae Dahuricae 1.2%-35.0% in weight.

Furthermore, this invention also provides a traditional Chinese medicine combination used for anti-EV71 or treatment and/or prevention on the diseases induced by EV71, and the said traditional Chinese medicine combination is prepared of the crude drugs as follows: Herba Houttyniae, Caulis Lonicerae, Radix Isatidis, Radix Sophorae Tonkinensis, Cyrtomium fortunei, Radix Angelicae Dahuricae, Rhizoma Paridis, Herba Artemisiae Annuae and Rhizoma Belamcandae.

Preferably, the weight percentages for the nine crude drugs in the said traditional Chinese medicine combination are as below: Herba Houttyniae 9.3%-46.7% in weight, Caulis Lonicerae 9.3%-37.3% in weight, Radix Isatidis 4.7%-35.0% in weight, Cyrtomium fortunei 2.3%-28.0% in weight, Radix Sophorae Tonkinensis 2.3%-23.3% in weight, Radix Angelicae Dahuricae 2.3%-23.3% in weight, Rhizoma Paridis 2.3%-23.3% in weight, Herba Artemisiae Annuae 2.3%-23.3% in weight, Rhizoma Belamcandae. 2.3%-23.3% in weight.

More preferably, the weight percentages for the nine crude drugs in the said traditional Chinese medicine combination are: Herba Houttyniae 14.0%-35.0% in weight, Caulis Lonicerae 11.7%-23.3% in weight, Radix Isatidis 9.3%-21.0% in weight, Cyrtomium fortunei 7.0%-18.7% in weight, Radix Sophorae Tonkinensis 4.7%-11.7% in weight, Radix Angelicae Dahuricae 4.7%-11.7% in weight, Rhizoma Paridis 4.7%-11.7% in weight, Herba Artemisiae Annuae 4.7%-11.7% in weight, Rhizoma Belamcandae. 4.7%-11.7% in weight.

Most preferably, the percentages by weight for the nine crude drugs in the said traditional Chinese medicine combination are: Herba Houttyniae 21.4% in weight, Caulis Lonicerae 17.9% in weight, Radix Isatidis 14.3% in weight, Cyrtomium fortunei 10.7% in weight, Radix Sophorae Tonkinensis 7.1% in weight, Radix Angelicae Dahuricae 7.1% in weight, Rhizoma Paridis 7.1% in weight, Herba Artemisiae Annuae 7.2% in weight, Rhizoma Belamcandae 7.2% in weight.

Among the uses above, preferably, the traditional Chinese medicine is prepared into granules.

In a further aspect, this invention provides a traditional Chinese medicine combination used for preparation of anti-EV71 drugs or the drugs for treatment and/or prevention of the diseases induced by EV71, and the traditional Chinese medicine combination is prepared of the crude drugs as follows: Herba Houttyniae, Caulis Lonicerae, Radix Isatidis and Radix Sophorae Tonkinensis.

Preferably, the weight percentages for the four crude drugs in the said traditional Chinese medicine combination are: Herba Houttyniae 4.7%-70.0% in weight, Caulis Lonicerae 4.7%-46.7% in weight, Radix Isatidis 2.3%-42.0% in weight, Radix Sophorae Tonkinensis 1.2% t-35.0% in weight.

Preferably, the said traditional Chinese medicine combination also contains other traditional Chinese medicines for eliminating heat evil and removing toxins; preferably, the said other traditional Chinese medicines for eliminating heat evil and removing toxins are selected from Cyrtomium fortunei, Radix Angelicae Dahuricae, Herba Andrographitis, Herba Taraxaci, Indigo Naturalis, Caulis Sargentodoxae, Rhizoma Paridis, Herba Artemisiae Annuae, Rhizoma Belamcandae, Brucea javanica, Herba Menthae Haplocalycis, Radix Platycodi and Radix Glycyrrhizae.

This invention also provides a traditional Chinese medicine combination used for preparation of anti-EV71 drugs or the drugs for treatments and/or prevention of the diseases induced by EV71, and the traditional Chinese medicine combination is prepared of the crude drugs as follows: Herba Houttyniae, Caulis Lonicerae, Radix Isatidis, Radix Sophorae Tonkinensis, Cyrtomium fortunei and Radix Angelicae Dahuricae.

Preferably, the weight percentages for the six crude drugs in the said traditional Chinese medicine combination are: Herba Houttyniae 4.7%-58.4% in weight, Caulis Lonicerae 4.7%-46.7% in weight, Radix Isatidis 2.3%-42.0% in weight, Radix Sophorae Tonkinensis 1.2%-35.0% in weight, Cyrtomium fortunei 1.9%-37.3% in weight, Radix Angelicae Dahuricae 1.2%-35.0% in weight.

Additionally, this invention also provides a traditional Chinese medicine combination used for preparation of anti-EV71 drugs or of the drugs for treatments and/or prevention of the diseases induced by EV71, and the said traditional Chinese medicine combination is prepared of the crude drugs as follows: Herba Houttyniae, Caulis Lonicerae, Radix Isatidis, Radix Sophorae Tonkinensis, Cyrtomium fortunei, Radix Angelicae Dahuricae, Rhizoma Paridis, Herba Artemisiae Annuae and Rhizoma Belamcandae.

Preferably, the weight percentages for the nine crude drugs in the said traditional Chinese medicine combination are: Herba Houttyniae 9.3%-46.7% in weight, Caulis Lonicerae 9.3%-37.3% in weight, Radix Isatidis 4.7%-35.0% in weight, Cyrtomium fortunei 2.3%-28.0% in weight, Radix Sophorae Tonkinensis 2.3%-23.3% in weight, Radix Angelicae Dahuricae 2.3%-23.3% in weight, Rhizoma Paridis 2.3%-23.3% in weight, Herba Artemisiae Annuae 2.3%-23.3% in weight, Rhizoma Belamcandae 2.3%-23.3% in weight.

More preferably, the weight percentages for the nine crude drugs in the said traditional Chinese medicine combination are: Herba Houttyniae 14.0%-35.0% in weight, Caulis Lonicerae 11.7%-23.3% in weight, Radix Isatidis 9.3%-21.0% in weight, Cyrtomium fortunei 7.0%-18.7% in weight, Radix Sophorae Tonkinensis 4.7%-11.7% in weight, Radix Angelicae Dahuricae 4.7%-11.7% in weight, Rhizoma Paridis 4.7%-11.7% in weight, Herba Artemisiae Annuae 4.7%-11.7% in weight, Rhizoma Belamcandae 4.7% in weight.

Most preferably, the weight percentages for the nine crude drugs in the said traditional Chinese medicine combination are: Herba Houttyniae 21.4% in weight, Caulis Lonicerae 17.9% in weight, Radix Isatidis 14.3% in weight, Cyrtomium fortunei 10.7% in weight, Radix Sophorae Tonkinensis 7.1% in weight, Radix Angelicae Dahuricae 7.1% in weight, Rhizoma Paridis 7.1% in weight, Herba Artemisiae Annuae 7.2% in weight, Rhizoma Belamcandae. 7.2% in weight. Among the above uses, preferably, the traditional Chinese medicine is prepared into granules.

In a further aspect, this invention also provides a traditional Chinese medicine combination used for anti-EV71 or treatment and/or prevention of the diseases induced by EV71, and the said traditional Chinese medicine combination is prepared of the crude drugs as follows: Herba Houttyniae, Caulis Lonicerae, Radix Isatidis and Radix Sophorae Tonkinensis.

Preferably, the weight percentages for the four crude drugs in the said traditional Chinese medicine combination are: Herba Houttyniae 4.7%-70.0% in weight, Caulis Lonicerae 4.7%-46.7% in weight, Radix Isatidis 2.3%-42.0% in weight, Radix Sophorae Tonkinensis 1.2%-35.0% in weight.

Preferably, the said traditional Chinese medicine combination also contains other traditional Chinese medicines for eliminating heat evil and removing toxins; preferably, the said other traditional Chinese medicines for eliminating heat evil and removing toxins are selected from Cyrtomium fortunei, Radix Angelicae Dahuricae, Herba Andrographitis, Herba Taraxaci, Indigo Naturalis, Caulis Sargentodoxae, Rhizoma Paridis, Herba Artemisiae Annuae, Rhizoma Belamcandae, Brucea javanica, Herba Menthae Haplocalycis, Radix Platycodi and Radix Glycyrrhizae.

This invention also provides a traditional Chinese medicine combination used for anti-EV71 or treatment and/or prevention of the diseases induced by EV71, and the said traditional Chinese medicine combination is prepared of the crude drugs as follows: Herba Houttyniae, Caulis Lonicerae, Radix Isatidis, Radix Sophorae Tonkinensis, Cyrtomium fortunei and Radix Angelicae Dahuricae.

Preferably, the weight percentages for the six crude drugs in the said traditional Chinese medicine combination are: Herba Houttyniae 4.7%-58.4% in weight, Caulis Lonicerae 4.7%-46.7% in weight, Radix Isatidis 2.3%-42.0% in weight, Radix Sophorae Tonkinensis 1.2%-35.0% in weight, Cyrtomium fortunei 1.9%-37.3% in weight, Radix Angelicae Dahuricae 1.2%-35.0% in weight.

Moreover, this invention also provides a traditional Chinese medicine combination used for anti-EV71 or treatment and/or prevention of the diseases induced by EV71, and the said traditional Chinese medicine combination is prepared of the crude drugs as follows: Herba Houttyniae, Caulis Lonicerae, Radix Isatidis, Radix Sophorae Tonkinensis, Cyrtomium fortunei, Radix Angelicae Dahuricae, Rhizoma Paridis, Herba Artemisiae Annuae and Rhizoma Belamcandae.

Preferably, the weight percentages for the nine crude drugs in the said traditional Chinese medicine combination are: Herba Houttyniae 9.3%-46.7% in weight, Caulis Lonicerae 9.3%-37.3% in weight, Radix Isatidis 4.7%-35.0% in weight, Cyrtomium fortunei 2.3%-28.0% in weight, Radix Sophorae Tonkinensis 2.3%-23.3% in weight, Radix Angelicae Dahuricae 2.3%-23.3% in weight, Rhizoma Paridis 2.3%-23.3% in weight, Herba Artemisiae Annuae 2.3%-23.3% in weight, Rhizoma Belamcandae. 2.3%-23.3% in weight.

More preferably, the weight percentages for the nine crude drugs in the said traditional Chinese medicine combination are: Herba Houttyniae 14.0%-35.0% in weight, Caulis Lonicerae 11.7%-23.3% in weight, Radix Isatidis 9.3%-21.0% in weight, Cyrtomium fortunei 7.0%-18.7% in weight, Radix Sophorae Tonkinensis 4.7%-11.7% in weight, Radix Angelicae Dahuricae 4.7%-11.7% in weight, Rhizoma Paridis 4.7%-11.7% in weight, Herba Artemisiae Annuae 4.7%-11.7% in weight, Rhizoma Belamcandae 4.7%-11.7% in weight.

Most preferably, the weight percentages for the nine crude drugs in the said traditional Chinese medicine combination are: Herba Houttyniae 21.4% in weight, Caulis Lonicerae 17.9% in weight, Radix Isatidis 14.3% in weight, Cyrtomium fortunei 10.7% in weight, Radix Sophorae Tonkinensis 7.1% in weight, Radix Angelicae Dahuricae 7.1% in weight, Rhizoma Paridis 7.1% in weight, Herba Artemisiae Annuae 7.2% in weight, Rhizoma Belamcandae 7.2% in weight.

The diseases induced by EV71 as mentioned above are selected from one or several of the diseases of hand-foot-mouth disease, herpangina, aseptic meningitis and encephalitis.

In a further aspect, this invention provides a method for anti-EV71 or treatment and/or prevention of the diseases induced by EV71, and the said method includes the administration of the traditional Chinese medicine combination as mentioned above in effective dose to the patients in need.

Preferably, the diseases induced by the said EV71 are selected from one or several of the diseases of hand-foot-mouth disease, herpangina, aseptic meningitis and encephalitis; more preferably, the said traditional Chinese medicine combination is prepared into granules.

It is found in this invention after large amount of experiments that the traditional Chinese medicine combination prepared of Herba Houttyniae, Caulis Lonicerae, Radix Isatidis and Radix Sophorae Tonkinensis as the raw drugs has significant anti-EV71 efficacy, which can thereby successively prevent and/or treat several diseases induced by the virus, including hand-foot-mouth disease, herpangina, aseptic meningitis and encephalitis.

Moreover, it is also found in this invention that the traditional Chinese medicine combination prepared of the raw drugs as below also shows certain anti-EV71 efficacy: Radix Isatidis 14.0%-37.3% in weight, Fructus Forsythiae Suspensae 4.7-25.7% in weight, Herba Pogostemonis 3.7%-11.7% in weight, Gypsum Fibrosum 3.3%-25.7 weight %, Rhizoma Phragmitis 3.7%-25.7% in weight, Radix Rehmanniae 3.7%-14.0% in weight, Rhizoma Acori Tatarinowii 3.7%-14.0% in weight, Radix Curcumae 3.7%-14.0% in weight, Rhizoma Anemarrhenae 3.7%-14.0% in weight.

It is indicated that the traditional Chinese medicine combination provided in this invention with natural plants as the raw drugs has significant inhibitory efficacy on EV71, the drugs or additives made by this combination have advantages of low toxicity, strong pharmacological action, safety and effectiveness, which open up new ways for preventing and treating EV71 induced diseases. It has important social and economic values and broad prospect for applications.

### BRIEF DESCRIPTIONS FOR THE ATTACHED FIGURES

The implementation program for this invention is illustrated in details with reference to the attached figures, among which:

SC: Sichuan prescription; GB: national standard prescription (Guobiao); negative: negative control; positive: positive control; d2, d3 or d4 (D2, D3 or D4): the second, third or fourth day; sc5: 5 mg/ml SC; sc10: 10 mg/ml SC; sc20: 20 mg/ml SC; gblO: lOmg/ml GB; gb20: 20 mg/ml GB; gb40: 40 mg/ml GB;

FIG. 1 shows the effects of SC on the vitality of Vero cells;

FIG. 2 shows the effects of GB on the vitality of Vero cells;

FIG. 3 shows the microscopic cell changes after the Sichuan prescription of anti-EV71 virus is added;

FIG. 4 shows the microscopic cell changes after the national standard prescription of anti-EV71 virus is added;

FIG. 5 shows the agar gel electrophoresis for virus proliferation after the Sichuan prescription of anti-EV71 virus is added;

FIG. 6 shows the agar gel electrophoresis for virus proliferation after the national standard prescription of anti-EV71 virus is added;

FIG. 7 shows the real-time PCR detection results for virus proliferation after the Sichuan prescription of anti-EV71 virus is added (20 times of TCID₅₀ EV71 virus);

FIG. 8 shows the results of real-time PCR detection for virus proliferation after the national standard prescription of anti-EV71 virus is added (20 times of TCID₅₀ EV71 virus);

### OPTIMAL WAY FOR IMPLEMENTING THE INVENTION

Further illustrations are carried out in details by combining specific implementation ways as below, and the examples are provided only to illustrate this invention but not to limit the scope of this invention.

### Example 1

This example provides the prescription of raw drugs for the traditional Chinese medicine combination (Sichuan prescription) and the preparation method as follows:

The raw drugs: Herba Houttyniae 21.4% in weight, Caulis Lonicerae 17.9% in weight, Radix Isatidis 14.3% in weight, Cyrtomium fortunei 10.7% in weight, Radix Sophorae Tonkinensis 7.1% in weight, Radix Angelicae Dahuricae 7.1% in weight, Rhizoma Paridis 7.1% in weight, Herba Artemisiae Annuae 7.2% in weight, Rhizoma Belamcandae. 7.2% in weight.

The nine traditional Chinese raw drugs are weighed according to the prescription. Firstly, Herba Houttyniae is subjected to decoction with water for 10˜40 minutes, followed by filtration. It is cooled after the filtrate is concentrated to a clear paste with a relative density of 1.05-1.20. Afterwards ethanol is added to a final ethanol content of 50˜80%. Keep still for 24˜48 hours and filter. Then ethanol is recovered from the filtrate, which is concentrated to a clear paste with a relative density of 1.05-1.25 for further use; Caulis Lonicerae and other seven raw drugs are subjected to decoction with water for twice, 2 hours once, followed by filtration. When the filtrate is concentrated to a relative density of 1.40, it is filtered and the filtrate is concentrated again to a clear paste with a relative density of 1.45.

The clear paste of herba houttyniae as mentioned above is added and fully mixed. It is further concentrated to a thick paste with a relative density of 1.10. The thick paste and the fine powder of sugar are granulated in a ratio of 1:3˜8. The combination is obtained after distribution.

### Example 2

This example provides the prescription of raw drugs for the traditional Chinese medicine combination (national standard prescription) and the preparation method as follows:

Raw drugs: Radix Isatidis 30.1% in weight, Fructus Forsythiae Suspensae 10.8% in weight, Gypsum Fibrosum 13.4% in weight, Rhizoma Anemarrhenae 5.8% in weight, Rhizoma Phragmitis 14.2% in weight, Radix Rehmanniae 7.5% in weight, Herba Pogostemonis 6.6% in weight, Acorns calamus L. 5.8% in weight, Radix Curcumae Wenyujin 5.8% in weight.

The nine traditional Chinese raw drugs are weighed according to the prescription. Firstly, Rhizoma Anemarrhenae is crushed into coarse powder. 80˜90% ethanol is added and the solution is heated for backstreaming and extraction for three times, 3 hours each time. Afterwards, it is filtered and the filtrate is subjected to decompressed recovery of ethanol and concentration. A clear paste with a relative density of 1.10 (50° C.) is obtained for further use. Radix Isatidis and other seven drugs are subjected to decoction with water for three times (volatile oil is collected simultaneously), 1 hour for the first time, 0.5 h for the second and the third time respectively. Filter and concentrate the filtrate to obtain the clear paste with a relative density of 1.16 (50° C.). The clear paste of Rhizoma Anemarrhenae as mentioned above is added, fully mixed and concentrated, and the clear paste with a relative density of 1.28˜1.30 (55˜60° C.) is obtained. The clear paste and proper amount of sugar powder are subjected to granulation. Proper amount of the collected volatile oil as mentioned above and food flavor are added after drying, and fully mixed for granulation. The final combination is obtained after distribution.

### Example 3

In vitro anti-EV71 test

This example tests the in vitro anti-EV71 efficacy of the traditional Chinese medicine combinations prepared in Example 1 and Example 2, with details shown below:

### 1. Materials and Methods

### 1. Materials

1.1 Experimental Drugs:

National standard prescription KBD-GB (Approval document number of State Food and Drug Administration Z20010127): 4.3 g crude drug/ml extract.

Sichuan prescription KBD-SC (Approval document number of State Food and Drug Administration Z51020073): 4.7 g crude drug/ml extract.

1.2 Cells: Vero cells (African green monkey kidney cells): provided by the National Key Laboratory of Newly Occurred Infectious Diseases in Hong Kong University.

1.3 Viruses: EV71/120F1/Shenzhen/2009, provided by the National Key Laboratory of Newly Occurred Infectious Diseases in Hong Kong University.

1.4 Basic conditions for the laboratory: the entire experimental procedure is finished in a grade-two bio-safe laboratory.

### 2. Experimental Methods:

2.1 Observations on Cytopathic Effects (CPE) and Determination of 50% Infective Dose of the Virus for Vero Cells (tcid₅₀)

Vero cells are inoculated in 96-well plates, 100 μl per well. After incubated at 37° C. and 5% CO₂ for 24 hours, the cells grow into a monolayer. A series of viral cultures in nine concentrations that are diluted in 10 fold are added. Eight wells are prepared for each concentration and the cell control is also set up. The cells are incubated at 37° C. in an incubator with 5% CO₂. CPE is observed after 72 hours. Read-Muench method is used to calculate the 50% infective dose of the virus for Vero cells (tcid₅₀).

2.2 Determination of Toxic Reactions of the Test Drug on the Cells with MTT Method

Vero cells are inoculated in 96-well plates, 100 μl per well. After incubated at 37° C. and 5% CO₂ for 24 hours, the cells grow into a monolayer. The supernatant is removed, and the test drugs in different dilution strengths are added. Eight wells are prepared for each concentration and the cell control is also set up. The cells are incubated at 37° C. in an incubator of 5% CO₂ and MTT is added for 3-hour staining after 72 hours. Afterwards the solution is removed and DMSO is added for dissolution for half an hour. The absorbance at OD570 nm is determined by using a enzyme-linked microplate reader. The maximal non-toxic concentration of the drug is determined according to the vitality of the cells.

2.3 Photographing of Microscopic Cell Changes

Vero cells are inoculated in 96-well plates, 100 μl per well. After incubated at 37° C. and 5% CO₂ for 24 hours, the cells grow into a monolayer. The drug is subjected to 10 fold dilution from the maximal non-toxic concentration and totally three concentrations are set up. Two wells are prepared for each concentration, 1 mL per well, and the cells are incubated at 37° C. and 5% CO₂ for 24 hours. The drug is removed, and 120 virus with a concentration of 20 times of TCID50 is added; the virus is removed after 1 hour, and 1 mL drug is added. The cells are incubated at 37° C. in an incubator with 5% CO₂. The cells are photographed on the second, third and fourth day of culture under the microscope respectively. The negative control is physiological saline and the positive control is treated by Tamiflu.

2.4 PCR and Real-Time PCR Detection for Virus Proliferation

Vero cells are inoculated in 96-well plates, 100 μl per well. After incubated at 37° C. and 5% CO₂ for 24 hours, the cells grow into a monolayer. The drug is subjected to 10 fold dilution from the maximal non-toxic concentration, and totally three concentrations are set up. Two wells are prepared for each concentration, 1 mL per well, and the cells are incubated at 37° C. and 5% CO₂ for 24 hours. The drug is removed, and 120 μl virus with a concentration of 20 times of TCID50 is added. The virus is removed after 1 hour, and 1 mL drug is added. The cells are incubated at 37° C. in an incubator with 5% CO₂. RNA is extracted from the supernatant at 24, 48 and 72 hours respectively. PCR and Real-time PCR are carried out to detect virus proliferation.

### 3. Results

3.1 Determination of TCID₅₀ for the virus: TCID₅₀ of the virus EV71/120F1/Shenzhen/2009 is 10^{5.125}/ml.

3.2 Determination of Drug Toxicity:

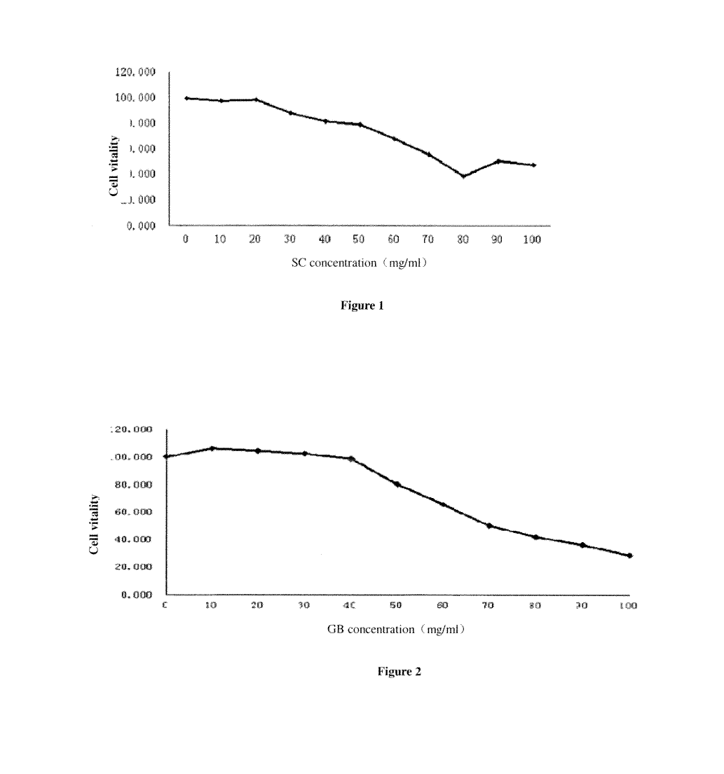

The maximal non-toxic concentration of Sichuan prescription KBD-SC (Z51020073) is 20 mg/ml. See FIG. 1;

The maximal non-toxic concentration of the national standard prescription KBD-GB (Z20010127) is 40 mg/ml. See FIG. 2.

3.3 Microscopic Cell Changes

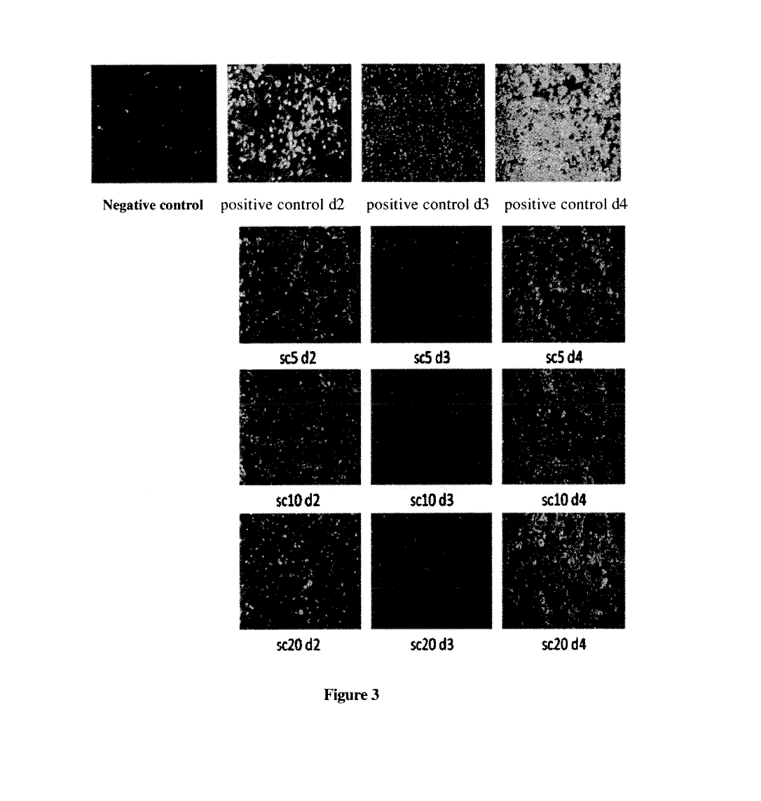

The microscopic cell changes for anti-EV71 by using Sichuan prescription are shown in FIG. 3. It can be found under the microscope that a considerable number of living cells can be seen on the third day of viral infection when the drug concentrations are 5 mg/ml, 10 mg/ml and 20 mg/ml respectively. The morphology of some living cells is abnormal when the drug concentration is 20 mg/ml, and the possibility of toxic effects of the drug on the cells can not be excluded.

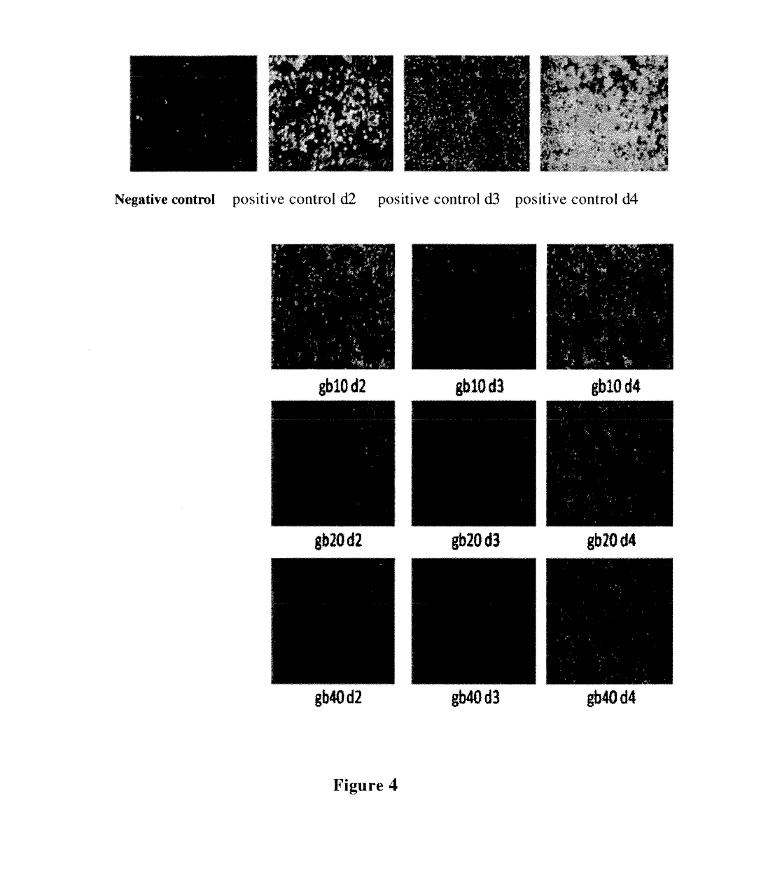

The microscopic cell changes for anti-EV71 by using national standard prescription are shown in FIG. 4. It can be found under the microscope that a considerable number of living cells can be seen on the third day of viral infection when the drug concentrations are 10 mg/ml and 20 mg/ml respectively. No living cell can be detected when the drug concentration reaches 40 mg/ml, which can be considered as the toxic effects of the drug on cells.

3.4 PCR Detection of Viral Proliferation at 24, 48 and 72 Hours

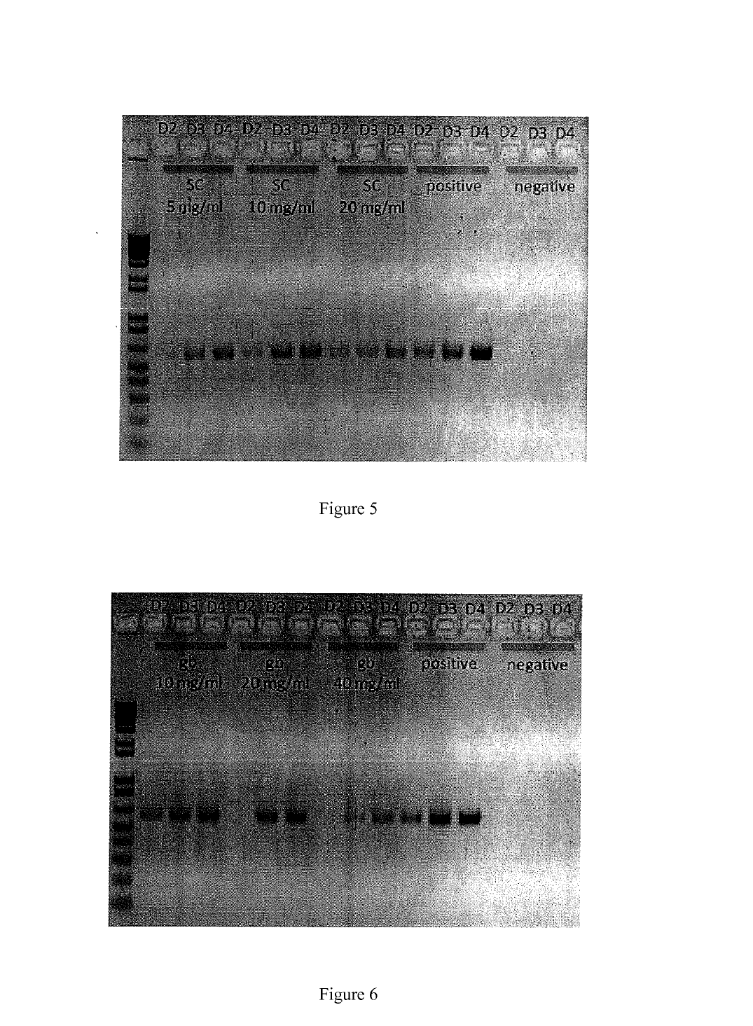

Agar gel electrophoresis is carried out to determine viral proliferation after Sichuan prescription is added, and the results are shown in FIG. 5. It can be found that Sichuan prescription has certain inhibitory effects on EV71 proliferation. The darker the electrophoretic band of PCR product in the figure is, the more the virus proliferates. The results show that viral proliferation can be inhibited on the third and fourth day of viral infection when the drug concentrations are 5 mg/ml, 10 mg/ml and 20 mg/ml, respectively.

Agar gel electrophoresis is carried out to determine viral proliferation after Sichuan prescription is added, the results are shown in FIG. 6. It can be found that national standard prescription has certain inhibitory effects on EV71 proliferation. The denser the electrophoretic band of PCR product is in the figure, the more the virus proliferates. The results show that viral proliferation can be inhibited on the third and fourth day of viral infection when the drug concentrations are 10 mg/ml and 20 mg/ml, respectively. The toxic effects of the drug on cells inhibit the viral proliferation when the drug concentration reaches 40 mg/ml.

3.5 Quantitative Detection of Viral Proliferation by Real-Time PCR

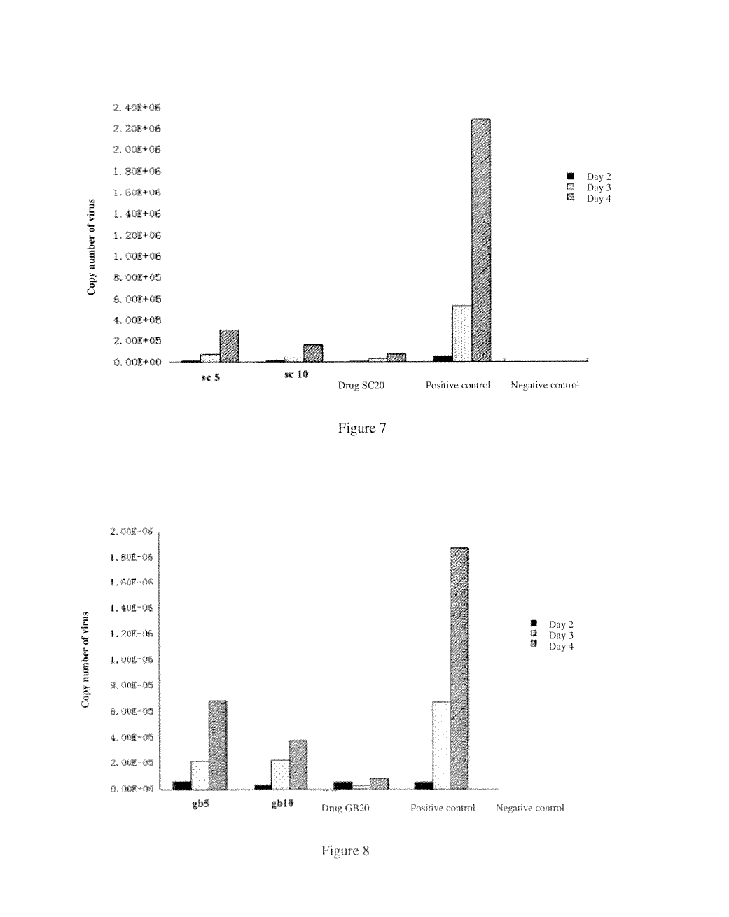

Real-time PCR is carried out to detect viral proliferation when Sichuan prescription is added. The results from real-time PCR (see FIG. 7 and Table 1) show that Sichuan prescription at different concentrations can inhibit EV71 proliferation. The drugs with concentrations of 5 mg/ml, 10 mg/ml and 20 mg/ml can significantly inhibit viral proliferation on the fourth day after viral infection.

TABLE 1
Real-time PCR detection for copy number of the virus after Sichuan
prescription is added

| | Copy number of the virus | | | | |
|---|---|---|---|---|---|
| | | | SC | | |
| | SC | SC | 20 mg/ | Positive | Negative |
| Time | (5 mg/ml) | (10 mg/ml) | (ml) | control | control |
| Day 2 | 163 × 10² | 133 × 10² | 89.80 × | 602 × 10² | 1.30 × 10² |
| | | | 10² | | |
| Day 3 | 786 × 10² | 549 × 10² | 356 × | 5300 × 10² | 1.00 × 10² |
| | | | 10² | | |
| Day 4 | 3080 × 10² | 1630 × 10² | 824 × | 22800 × 10² | 1.00 × 10² |
| | | | 10² | | |

Real-time PCR detection is carried out for viral proliferation when national standard prescription is added. The results from real-time PCR (see FIG. 8 and Table 2) show that national standard prescriptions with different concentrations can inhibit EV71 proliferation at early stages. The drug with concentrations of 10 mg/ml and 20 mg/ml can significantly inhibit viral proliferation on the fourth day after viral infection. The toxic effects of the drug on cells inhibit the viral proliferation when the drug concentration reaches 40 mg/ml.

TABLE 2
Real-time PCR detection for copy number of the virus
after national standard prescription is added

| | Copy number of the virus | | | | |
|---|---|---|---|---|---|
| | GB | | | | |
| | (10 mg/ | GB | GB | Positive | Negative |
| Time | ml) | (20 mg/ml) | (40 mg/ml) | control | control |
| Day | 579 × | 296 × 10² | 54.10 × 10² | 507 × 10² | 1.00 × 10² |
| 2 | 10² | | | | |
| Day | 2170 × | 2200 × 10² | 257 × 10² | 6710 × 10² | 1.00 × 10² |
| 3 | 10² | | | | |
| Day | 6810 × | 3740 × 10² | 809 × 10² | 18700 × 10² | 1.00 × 10² |
| 4 | 10² | | | | |

### 4. Conclusions

The above results indicate that Sichuan prescription KBD-SC and national standard prescription KBD-GB effectively inhibit viral proliferation at early stages of EV71 infection. The toxic reactions of Sichuan prescription are more significant than those of national standard prescription KBD-GB.

Having thus described the invention, it is now claimed:

1. A composition used for anti-EV71 or treatment and/or prevention of the diseases induced by EV71, the composition comprising:
4.7% to 70.0% by weight Herba Houttyniae,
4.7% to 46.7% by weight Caulis Lonicerae,
2.3% to 42.0% by weight Radix Isatidis; and,
1.2% to 35.0% by weight Radix Sophorae Tonkinensis.

2. The composition of claim 1, wherein the composition further comprises:
at least of the group selected from Cyrtomium fortunei, Radix Angelicae Dahuricae, Herba Andrographitis, Herba Taraxaci, Indigo Naturalis, Caulis Sargentodoxae, Rhizoma Paridis, Herba Artemisiae Annuae, Rhizoma Belamcandae, Brucea javanica, Herba Menthae Haplocalycis, Radix Platycodi and Radix Glycyrrhizae.

3. The composition of claim 1, wherein the composition further comprises:
1.9% to 37.3% by weight Cyrtomium fortunei; and,
1.2% to 35.0% by weight Radix Angelicae Dahuricae, wherein the Herba Houttyniae is 4.7% to 58.4% by weight.

4. The composition of claim 3, wherein the composition further comprises:
2.3% to 23.3% by weight Rhizoma Paridis,
2.3% to 23.3% by weight Herba Artemisiae Annuae; and,
2.3% to 23.3% by weight Rhizoma Belamcandae, wherein the Herba Houttyniae is 9.3% to 46.7% by weight, Caulis Lonicera is 9.3% to 37.3% by weight, Radix Isatidis is 4.7% to 35.0% by weight, Radix Sophorae Tonkinensis is 2.3% to 23.3% by weight, Cyrtomium fortune is 2.3% to 28.0% by weight, Radix Angelicae Dahuricae is 2.3% to 23.3% by weight, and Rhizoma Paridis is 2.3% to 23.3% by weight.

5. A method of treating EV71 and diseases induced by EV71 by administering the composition of claim 1.

6. (canceled)

7. The method of claim 5, wherein the diseases induced by EV71 are selected from the group comprising hand-foot and mouth disease, herpangina, aseptic meningitis and encephalitis wherein the composition is prepared into granules.

8. (canceled)

9. (canceled)

10. The composition of claim 4, wherein Herba Houttyniae is 14.0%-35.0% by weight, Caulis Lonicerae is 11.7%-23.3% by weight, Radix Isatidis is 9.3%-21.0% by weight, Cyrtomium fortunei is 7.0%-18.7% by weight, Radix Sophorae Tonkinensis is 4.7%-11.7% by weight, Radix Angelicae Dahuricae is 4.7%-11.7% by weight, Rhizoma Paridis is 4.7%-11.7% by weight, Herba Artemisiae Annuae is 4.7%-11.7% by weight, and Rhizoma Belamcandae is 4.7%-11.7% by weight.

11. The composition of claim 10, wherein Herba Houttyniae is 21.4% by weight, Caulis Lonicerae is 17.9% by weight, Radix Isatidis is 14.3% by weight, Cyrtomium fortune is 10.7% by weight, Radix Sophorae Tonkinensis is 7.1% by weight, Radix Angelicae Dahuricae is 7.1% by weight, Rhizoma Paridis is 7.1% by weight, Herba Artemisiae Annuae is 7.2% by weight, and Rhizoma Belamcandae is 7.2% by weight.
